(19) United States
(12) Patent Application Publication
(10) Pub. No.: US 2008/0199533 A1
(43) Pub. Date: Aug. 21, 2008

(54) Cosmetic and skin care formulation containing pearls, teas and fruits

(76) Inventor: Dan M. DeLaRosa, New York, NY (US)

Correspondence Address: Dan M. DeLaRosa, Esq., Suite 24C, 300 E 77th Street, New York, NY, 10075, US

(21) Appl. No.: 12/070,025

(22) Filed: Feb. 15, 2008

Related U.S. Application Data
(60) Provisional application No. 60/902,376, filed on Feb. 21, 2007.

(51) Int. Cl. A61K 35/56; A61Q 19/00

(52) U.S. Cl. 424/547

(57) ABSTRACT

The present invention relates to a cosmetic and skin care formulation comprising: at least one pearl selected from a group consisting essentially of cultured pearls, Japanese natural pearls, cultured pearls, freshwater pearls, salt water pearls, keshi pearls, mabe pearls and mixtures thereof; at least one tea selected from a group consisting essentially of white tea, black tea, green tea, red tea, oolong tea, herbal tea and mixtures thereof; and at least one fruit selected from a group consisting essentially of miracle fruit, Barbados cherry, peanut butter fruit, sugar apple, wampi, imbé, Spanish lime, langsat, black sapote, papaya, velvet apple, mabolo, soursop, guanabana, mangosteen, okari nut, passion fruit, rambutan, banana, plantain, mango, sapodilla, red banana, canistel, egg fruit, citron, pummelo, monstera, green papaya, pineapple, durian, star fruit, coconut, Jake fruit, pomegranate and mixtures thereof.

### RELATED APPLICATION

This application is related to U.S. Provisional Application Ser. No. 60/902,376, entitled “Cosmetic and Skin Care Formulation Containing Tri-Pearl Complex” which was filed on Feb. 21, 2007.

### BACKGROUND OF THE INVENTION

1. Field of the Invention

This invention relates to cosmetic and skin care formulations and related methods of manufacture and more particularly, a cosmetic and skin care formulation comprising pearls, teas and fruits.

2. Description of the Related Art

There are various skin care products that contain fresh water pearls and/or one or more types of teas. These products are used in various forms of moisturizers, exfoliants and other skin care applications. Pearls have been used in numerous skin care and beauty products. Such formulation use some form of crushed pearls such as pearl powder. Crushed pearls have been used in China for skin care and in traditional Chinese medicine as an anti-inflammatory and detoxification agent. Such crushed pearls or pearl powder is finely milled powder from quality fresh water pearls. It is naturally compatible and easily absorbed by the body and the skin. It is also been proven safe to ingest and apply to the skin directly. All of the pearl formulations in the marketplace are topically applied to the skin; usually whereby the pearl composition is applied by the hand or fingers from a container such as a jar.

The present formulation uses a multi-pearl or multi-tea or multi-fruit complex in a combination skin care and cosmetic product. The present invention uses, in one embodiment, the combination of fresh water pearls, black pearls and cultured pearls. In another embodiment, the present invention provides for formulations that contain at least three types of teas that are used both as a skin care and cosmetic product.

### SUMMARY OF THE INVENTION

The present invention relates to a cosmetic and skin care formulation comprising: at least one pearl, at least one vitamin, and wherein said pearl provides for a source of proteins and minerals. In another embodiment, the formulation further comprises peptides, and/or anti-oxidants, and/or anti-aging agent and/or teas. In still another embodiment, the tea is selected from a group consisting essentially of white tea, red tea, black tea, oolong tea and mixtures thereof.

In a further embodiment, the pearl is selected from a group consisting essentially of black pearl, cultured pearl, freshwater pearl and mixtures thereof. In yet a further embodiment, the formulation further comprising at least one humectant substance, said humectant substance is selected from a group consisting of glycerin, glycols, butylene glycol, propylene glycol, pentylene glycol, polyquarternium-7, and extracts thereof and mixtures thereof.

In another embodiment, the present invention relates to a cosmetic and skin care formulation comprising: at least one tea, at least one humectant, and at least one emollient.

In a further embodiment, the invention provides for a method of manufacturing a skin care and cosmetic formulation, said method comprises admixing at least one pearl, at least one vitamin, and wherein said pearl provides for a source of proteins and minerals.

The present invention can work with any types of pearls and any types of teas. In a further embodiment, the present invention provides for a cosmetic and skin care formulation comprising: at least one pearl selected from a group consisting essentially of cultured pearls, Japanese natural pearls, cultured pearls, freshwater pearls, salt water pearls, keshi pearls, mabe pearls and mixtures thereof; at least one tea selected from a group consisting essentially of white tea, black tea, green tea, red tea, oolong tea, herbal tea and mixtures thereof; and at least one fruit selected from a group consisting essentially of miracle fruit, Barbados cherry, peanut butter fruit, sugar apple, wampi, imbé, Spanish lime, langsat, black sapote, papaya, velvet apple, mabolo, soursop, guanabana, mangosteen, okari nut, passion fruit, rambutan, banana, plantain, mango, sapodilla, red banana, canistel, egg fruit, citron, pummelo, monstera, green papaya, pineapple, durian, star fruit, coconut, Jake fruit, pomegranate and mixtures thereof.

In another further embodiment, the formulation further comprises vitamins, minerals, peptides, anti-oxidants, anti-aging agents, humectants and/or emollients. In still a further embodiment, the humectant is selected from a group consisting essentially of glycerin, glycols, butylene glycol, propylene glycol, pentylene glycol, polyquarternium-7, and extracts thereof and mixtures thereof. In yet a further embodiment, the emollient is selected from a group consisting essentially of sweet almond oil, Squalene, apricot oil, safflower oil, sunflower oil, oils and mixtures thereof.

In still yet a further embodiment, the present invention provides for a cosmetic and skin care formulation comprising: at least one pearl selected from a group consisting essentially of cultured pearls, Japanese natural pearls, cultured pearls, freshwater pearls, salt water pearls, keshi pearls, mabe pearls and mixtures thereof; at least one humectant; and at least one emollient.

In another further embodiment, the formulation further comprises an exfoliating agent selected from a group consisting essentially of pearl, rice, polymers and mixtures thereof.

In another embodiment, the present invention provides for a cosmetic and skin care formulation comprising: at least one tea selected from a group consisting essentially of white tea, black tea, green tea, red tea, oolong tea, herbal tea and mixtures thereof; and at least one humectant and said humectant is selected from a group consisting essentially of glycerin, glycols, butylene glycol, propylene glycol, pentylene glycol, polyquarternium-7, and extracts thereof and mixtures thereof.

### DETAILED DESCRIPTION OF THE INVENTION

As required, detailed embodiments of the present invention are disclosed herein; however, it is to be understood that the disclosed embodiments are merely exemplary of the invention that may be embodied in various forms. Therefore, specific functional details disclosed herein are not to be interpreted as limiting, but merely as a basis for the claims and as a representative basis for teaching one skilled in the art to employ the present invention in various ways.

In one embodiment, the present invention relates to a cosmetic or skin care formulation comprising at least one pearl and the pearl is selected from a group consisting essentially of natural pearls, cultured pearls, Japanese cultured pearls, freshwater pearls, salt water pearls, keshi pearls, mabe pearls and mixtures thereof. Natural pearls are formed by nature without instigation from human beings.

Natural pearls are formed around an irritant such as a grain of sand, apiece of coral or seashell. Cultured pearls are generally formed the same way as natural pearls and the only difference is the irritant is introduced by man (the nucleus is inserted into the oyster by man) and the oysters are raised in a controlled environment. Japanese cultured pearls (including akoya pearls) are generally smaller in size than south sea pearls and colors include white, cream, gold and rose. Freshwater cultured pearls are farmed in freshwater and grown in mussels. Freshwater pearls cultivating techniques usually produce twenty or more pearls in one oyster, and it only takes one and a half years to two years to harvest. Saltwater cultured pearls are farmed in saltwater and grown in oysters. Unlike saltwater pearls, only one pearl is grown per oyster and needs two years and a half to get good harvest. The colors of the saltwater pearls are white, cream, yellow, gold, silver, rose and black. Countries that are producers are Burma, Indonesia, Thailand, Philippines, Australia and Tahiti. Keshi pearls are very small pearl byproducts of the pearl culturing process. If debris enters an oyster while it's open during the harvesting process, the oyster covers the debris with nacre, thus creating keshi pearls. Mabe pearls are created when human attaches a plastic dome to the inside shell of an oyster, which reacts by covering the dome with nacre.

In another embodiment, the present invention relates to a cosmetic or skin care formulation comprising at least one tea and the tea is selected from a group consisting essentially of white tea, black tea, green tea, red tea, oolong tea, herbal tea and mixtures thereof.

Black teas are oxidized; this means that the leaves are exposed to the air for a precise length of time in order to allow natural chemical reactions to occur. These reactions change the leaves' color from green to copper and intensify their flavor until it is rich and complex. Earl Grey, English Breakfast and Darjeeling are some examples of black teas.

Green teas are not oxidized; they are merely withered and dried. Green teas include Lung Ching, Bancha and Sencha. Green tea also has HGCG; the most powerful antioxidant known.

Oolong teas are a combination of green and black leaves. They are partly fermented and stronger than green teas, but are more delicate than the fully fermented black teas. Oolong tea, like black tea goes through a withering stage (wilting). The difference is the oolong tea, goes through a shorter stage and the leaves are fired directly after that to prevent continued oxidation (fermentation.) The leaves can range from being almost black to dark green depending on when oxidation is stopped. The longer the leaves are oxidized the closer to black tea they will become. Black Dragon and Pouchong (Pao-Chung) are samples of oolong teas.

China whites, produced exclusively in China and highly prized, are simply steamed and dried. White tea is the least processed tea. The leaves are picked early in the year while the tiny white hairs are still visible on the leaves and the bud is still closed. Only the top leaf and a bud are picked from the plant. The leaves are then allowed to dry in the sun; they are not steamed or pan fired like green tea. White teas include Flowery White Pekoe, Silver Needles and Noble Beauty.

Herbal teas are often called “tisanes” or “infusions”. Chamomile and Peppermint are just two of the many herbal teas available today. Red tea is also a sample of herbal teas and derived from the South African herb Roobios.

In another embodiment, the present invention relates to a cosmetic or skin care formulation comprising at least one exotic fruit and the fruit is selected from a group consisting essentially of miracle fruit, Barbados cherry, peanut butter fruit, sugar apple, wampi, imbé, Spanish lime, langsat, black sapote, papaya, velvet apple, mabolo, soursop, guanabana, mangosteen, okari nut, passion fruit, rambutan, banana, plantain, mango, sapodilla, red banana, canistel, egg fruit, citron, pummelo, monstera, green papaya, pineapple, durian, star fruit, coconut, Jake fruit, pomegranate and mixtures thereof.

In one embodiment, the formulation is manufactured by selecting at least one pearl, selecting at least one tea and selecting at least one fruit and admixing the pearl, tea and fruit.

In another embodiment, a skin care formulation is prepared and at least one pearl or one tea or one fruit is selected and admixed with the prepared formulation.

In a further embodiment, the formulation may further comprise an herbal extract selected from a group consisting essentially of angelica extract, ashwaganda extract, astragalus extract, bilberry extract, black cohosh extract, blue cohosh extract, blessed thistle extract, burdock extract, California poppy extract, cactus extract, celandine extract, chamomile extract, chaparral extract, chaste tree extract, chickweed extract, cleavers extract, coltsfoot extract, corn silk extract, cramp bark extract, damiana extract, dandelion extract, devil's claw extract, dong quai extract, echinacea extract, sib ginseng extract, eyebright extract, false unicorn root extract, fenugreek extract, ginkgo extract, ginseng root extract, goldenrod extract, goldenseal extract, gotu kola extract, horsetail extract, hyssop extract, kava kava extract, lady's mantle extract, licorice root extract, milk thistle extract, motherwort extract, nettle extract, oats extract, Oregon grape root extract, osha root extract, passionflower extract, pau d'arco extract, plantain extract, red clover extract, red root extract, reishi mushroom extract, sarsaparilla extract, saw palmetto extract, St. John's wort extract, schisandra berry extract, shepherds purse extract, skullcap extract, slippery elm extract, usnea lichen extract, uva ursi extract, valerian extract, wild cherry extract, wild yam extract, wormwood extract, yellow dock extract, yucca root extract and mixtures thereof. In a further embodiment, the herbal extract of the present invention also uses botanical extracts.

In still a further embodiment, the formulation comprising an aromatherapeutical substance which comprises essential oils and the essential oils can be selected from a group consisting essentially of absinthe oil, African bluegrass oil, ajowan oil, anethi oil, angelica root oil, anise star oil, arborvitae wild oil, armoise mugwort oil, asafetida oil, Australian balm oil, basil oil, benzoin oil, bergamot calabrian oil, betel leaf oil, birch sweet oil, birch tar oil, black currant-seed oil, blood orange oil, buchu oil, cabreuva oil, cade oil, cajeput oil, calamus root oil, camphor oil, cananga oil, caraway oil, cardamom oil, carrotseed oil, cassia oil, cedarleaf oil cedarwood atles oil, cedarwood Chinese oil, cedarwood Himalayan oil, cedarwood texas oil, cedarwood Virginian oil, celery seed oil, chamomile German oil, chamomile moroc oil, chamomile roman oil, chaulmogra oil, chenopodium oil, chilly seed oil, cinnamon bark oil, cinnamon leaf oil, citral oil, citronelia Ceylon oil, clarysage oil, clementine oil, clove bud oil, clove leaf oil, cognac oil, copaiba balsam oil, coriander oil, costus root oil, coffee oil, cumin oil, curcuma oil, curry leaf oil, cypress Australian blue oil, cypress French cypriol oil, davana oil, dill seed oil, dill weed oil, elemi oil, emu oil, eucalyptus oil, fennel bitter oil, fenugreek oil, fir balsam oil, fir needle oil, flax seed organic oil, frankincense oil garlic oil, galbanum oil, ginger oil, ginger grass oil, gaia oil, grapefruit pink oil, grapefruit white oil, guaiacwood oil, helichrysum oil, horseradish oil, hydacheim oil, hyssop oil, jamarosa root oil, Himalayan wild oil, karanj seed oil, kanuka oil, katrafay oil, key lime oil, kunzea oil, kukuinut oil, lantana oil, laurel leaf oil, lemon oil, lemongrass oil, lemon eucalyptus oil, lemon myrtle oil, lemon 5 fold oil, lican oil, lime oil, litsea cubeba oil, lovage leaf oil, mace oil, mandarin oil, manuka oil, marjoram oil, Melissa leaf oil, menthol liquid oil, mullein oil, myrrh oil, myrtle oil, neem oil, neroli oil, nerolina Australian oil, niaouli oil, noni oil, nutmeg oil, oat oil, onion oil, opoponax oil, orange bitter oil, orange sweet oil, origanum oil, orange 5 fold oil, palmarosa oil, parsley oil, patchouli oil, pennyroyal oil, pepper oil, peppermint oil, peru balsam oil, petitgrain oil, pimento leaf oil, pine long leaf oil, pine scotch oil, pine commercial oil, pomegranate oil, pomouwood oil, ravensara wild oil, rosalina Australian oil, rose geranium oil, rosemary oil, rosewood oil, rue oil, sage Dalmatian oil, sandalwood oil, savine oil, savory winter oil, sea buckthorn oil, spearmint oil, spikenard oil, spruce oil, sugandh kokila oil, sumac oil, tagetes oil, tangerine oil, tansy blue oil, tarragon oil, tangerine 5 fold oil, tea tree oil, tea tree lemon oil, thyme oil, tolu balsam oil, turmeric oil, valerian root oil, vanilla 10 fold oil, vetivewr oil, wintergreen oil, wild verbena oil, wormwood oil, yarrow oil, ylang ylang oil and mixtures thereof.

Therefore, while the embodiments of the present invention disclosed herein are presently considered to be preferred, various changes and modifications can be made without departing from the spirit and scope of the invention. The scope of the invention is indicated in the appended claims, and all changes that come within the meaning and range of equivalents are intended to be embraced therein.

What is claimed is:

1. A cosmetic and skin care formulation comprising:
at least one pearl selected from a group consisting essentially of cultured pearls, Japanese natural pearls, cultured pearls, freshwater pearls, salt water pearls, keshi pearls, mabe pearls and mixtures thereof;
at least one tea selected from a group consisting essentially of white tea, black tea, green tea, red tea, oolong tea, herbal tea and mixtures thereof; and
at least one fruit selected from a group consisting essentially of miracle fruit, Barbados cherry, peanut butter fruit, sugar apple, wampi, imbé, Spanish lime, langsat, black sapote, papaya, velvet apple, mabolo, soursop, guanabana, mangosteen, okari nut, passion fruit, rambutan, banana, plantain, mango, sapodilla, red banana, canistel, egg fruit, citron, pummelo, monstera, green papaya, pineapple, durian, star fruit, coconut, Jake fruit, pomegranate and mixtures thereof.

2. The formulation of claim 1 further comprising peptides.

3. The formulation of claim 1 further comprising anti-oxidants.

4. The formulation of claim 1 further comprising vitamins and minerals.

5. The formulation of claim 1 further comprising at least one humectant.

6. The formulation of claim 5 wherein said humectant is selected from a group consisting essentially of glycerin, glycols, butylene glycol, propylene glycol, pentylene glycol, polyquarternium-7, and extracts thereof and mixtures thereof.

7. The formulation of claim 1 further comprising at least one emollient.

8. The formulation of claim 7 wherein said emollient is selected from a group consisting essentially of sweet almond oil, Squalene, apricot oil, safflower oil, sunflower oil, oils and mixtures thereof.

9. The formulation of claim 1 further comprising an anti-aging agent.

10. A cosmetic and skin care formulation comprising:
at least one pearl selected from a group consisting essentially of cultured pearls, Japanese natural pearls, cultured pearls, freshwater pearls, salt water pearls, keshi pearls, mabe pearls and mixtures thereof;
at least one humectant; and at least one emollient.

11. The formulation of claim 10 further comprising at least one tea selected from a group consisting essentially of white tea, black tea, green tea, red tea, oolong tea, herbal tea and mixtures thereof

12. The formulation of claim 10 further comprising at least one peptide.

13. The formulation of claim 10 further comprising at least one vitamin and at least one mineral.

14. The formulation of claim 10 further comprising at least one exfoliating agent.

15. The formulation of claim 14 wherein said exfoliating agent is selected from a group consisting essentially of pearl, rice, polymers and mixtures thereof.

16. The formulation of claim 14 at least one fruit selected from a group consisting essentially of miracle fruit, Barbados cherry, peanut butter fruit, sugar apple, wampi, imbé, Spanish lime, langsat, black sapote, papaya, velvet apple, mabolo, soursop, guanabana, mangosteen, okari nut, passion fruit, rambutan, banana, plantain, mango, sapodilla, red banana, canister, egg fruit, citron, pummelo, monstera, green papaya, pineapple, durian, star fruit, coconut, Jake fruit, pomegranate and mixtures thereof.

17. A cosmetic and skin care formulation comprising: at least one tea selected from a group consisting essentially of white tea, black tea, green tea, red tea, oolong tea, herbal tea and mixtures thereof; and at least one humectant and said humectant is selected from a group consisting essentially of glycerin, glycols, butylene glycol, propylene glycol, pentylene glycol, polyquarternium-7, and extracts thereof and mixtures thereof.

18. The formulation of claim 17 further comprising peptides, vitamins and minerals.

19. The formulation of claim 17 wherein said formulation is used as a cleanser.

20. The formulation of claim 17 further comprises admixing at least one anti-aging agent.
